(19) United States
(12) Patent Application Publication
(10) Pub. No.: US 2013/0225981 A1
(43) Pub. Date: Aug. 29, 2013

(54) POSITION DETECTING APPARATUS OF CAPSULE ENDOSCOPE, CAPSULE ENDOSCOPE SYSTEM AND COMPUTER READABLE RECORDING MEDIUM

(71) Applicant: Olympus Medical Systems Corp., (US)

(72) Inventor: Jun HASEGAWA, Tokyo (JP)

(73) Assignee: OLYMPUS MEDICAL SYSTEMS CORP., Tokyo (JP)

(21) Appl. No.: 13/770,263

(22) Filed: Feb. 19, 2013

Related U.S. Application Data
(63) Continuation of application No. PCT/JP2012/052759, filed on Feb. 7, 2012.

(30) Foreign Application Priority Data
Mar. 2, 2011 (JP) 2011-045685

(51) Int. Cl. A61B 5/06; A61B 1/04

(52) U.S. Cl. CPC A61B 5/061; A61B 1/041
USPC 600/424

(57) ABSTRACT

A position detecting apparatus of a capsule endoscope includes a receiving antenna unit that receives a wireless signal transmitted together with an image data signal from the capsule endoscope in a subject via a plurality of receiving antennas, a correlation level calculating unit that calculates a correlation level between the image received by the receiving antennas and an image received immediately before the image was received, a determining unit that determines whether a position and/or direction of the capsule endoscope is changed, based on the correlation level calculated by the correlation level calculating unit, and an estimating unit that estimates the position and/or direction with respect to the position of the capsule endoscope at the time the image was captured if the determining unit determines that the position and/or direction of the capsule endoscope is changed.

### CROSS REFERENCES TO RELATED APPLICATIONS

This application is a continuation of PCT international application Ser. No. PCT/JP2012/052759 filed on Feb. 7, 2012 which designates the United States, incorporated herein by reference, and which claims the benefit of priority from Japanese Patent Application No. 2011-045685, filed on Mar. 2, 2011, incorporated herein by reference.

### BACKGROUND OF THE INVENTION

1. Field of the Invention

The present invention relates to a position detecting apparatus for receiving a wireless signal transmitted from a capsule endoscope in a subject by a receiving device outside the subject and detecting a position of the capsule endoscope based on the received wireless signal, and relates to a capsule endoscope system.

2. Description of the Related Art

In the related art, in the field of endoscopes, a capsule endoscope in which an imaging function, a wireless communication function, and the like are incorporated into a capsule-shaped housing that is formed in such a size that it can be inserted in the digestive tract of a subject such as a patient is known. This capsule endoscope moves along the inside of the subject, such as the digestive tract, according to a peristaltic motion or the like after it is swallowed from the mouth of the subject. Moreover, the capsule endoscope captures sequentially the images of the inside of the subject to generate image data and wirelessly transmits the image data sequentially.

The image data transmitted wirelessly from the capsule endoscope in this manner is received by a receiving device provided outside the subject, and the image data received by the receiving device is stored in a memory included in the receiving device. After an examination ends, the image data stored in the memory of the receiving device is imported in an image display device. An observer such as a doctor or a nurse observes an organ image or the like displayed by the image display device and performs a diagnosis on the subject.

Since the capsule endoscope moves along the body cavity according to a peristaltic motion or the like, it is necessary to correctly recognize the position inside the body cavity that the image data transmitted by the capsule endoscope is captured.

Thus, a capsule endoscope system that detects the position inside a subject, of a capsule endoscope based on a strength signal of a wireless signal transmitted from the capsule endoscope is disclosed (Japanese Laid-open Patent Publication No. 2006-288808 and Japanese Laid-open Patent Publication No. 2007-000608).

Moreover, a capsule endoscope which includes a sensor that collects internal information of a subject and which detects the position or the like inside a subject, of the capsule endoscope from information collected by the sensor is disclosed (Japanese National Publication of International Patent Application No. 2010-524557).

### SUMMARY OF THE INVENTION

A position detecting apparatus of a capsule endoscope according to one aspect of the present invention includes: a receiving antenna unit that receives a wireless signal transmitted together with an image data signal from the capsule endoscope in a subject via a plurality of receiving antennas; a first storage unit that stores a first image received by the receiving antennas; a second storage unit that stores a second image received subsequently to the first image; a correlation level calculating unit that calculates a correlation level between the first image stored in the first storage unit and the second image stored in the second storage unit; a determining unit that determines whether a position and/or a direction of the capsule endoscope is changed, based on comparison of the correlation level between the first and second images calculated by the correlation level calculating unit with a specified threshold value; an estimating unit that estimates the position and/or direction of the capsule endoscope at the time the second image was captured by an imaging unit of the capsule endoscope if the determining unit determines that the correlation level is smaller than the specified threshold value; and a third storage unit that stores the second image stored in the second storage unit in association with information of the position and/or direction estimated by the estimating unit.

A capsule endoscope system according to another aspect of the present invention includes: a capsule endoscope that acquires image data of a subject; the position detecting apparatus that receives the image data transmitted from the capsule endoscope and estimates the position and direction of the capsule endoscope when the position and/or direction of the capsule endoscope is determined to be changed; and an image display unit that acquires the image data and position information of the image data from the position detecting apparatus and displays the acquired image data and position information.

According to still another aspect of the present invention, a non-transitory computer readable recording medium storing an executable position determination program is presented. The position determination program causes a processor of a position detecting apparatus that receives image data transmitted from a capsule endoscope in a subject and estimates a position and a direction of the capsule endoscope in which the received image data was captured, to execute: acquiring a wireless signal which is transmitted from the capsule endoscope and received by a plurality of receiving antennas of a receiving antenna unit; extracting an image from the wireless signal received by the receiving antennas; calculating a correlation level between the extracted image and an image received immediately before the extracted image was received; determining whether the position and/or direction of the capsule endoscope is changed, based on the calculated correlation level by the correlation level calculating procedure; and estimating the position and/or direction of the capsule endoscope in which the image was captured if the position and/or direction of the capsule endoscope is determined to be changed by the determining procedure.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an overall configuration of a capsule endoscope system that uses a receiving device according to a first embodiment of the present invention;

FIG. 2 is a cross-sectional view illustrating an overall internal configuration of a capsule endoscope;

FIG. 3 is a block diagram illustrating an overall configuration of the receiving device according to the first embodiment of the present invention;

FIG. 4 is a diagram illustrating the relation between images captured by the capsule endoscope and the correlation level;

FIG. 5A is a schematic diagram for describing detection of the position of a capsule endoscope;

FIG. 5B is a schematic diagram in which the region of FIG. 5A is divided in quarters in xyz directions;

FIG. 6 is a diagram illustrating the component of an electromagnetic field at an optional position about an antenna (a circular coil antenna is used) of a capsule endoscope;

FIG. 7 is a diagram illustrating the attenuation of an electromagnetic field propagating in a medium;

FIG. 8 is a diagram illustrating the relation between an electric field generated by a capsule endoscope and the orientation of one receiving antenna of a receiving antenna unit;

FIG. 9A is an example of the trajectory of a capsule endoscope, within a subject, displayed by an image display device;

FIG. 9B is another example of the trajectory of a capsule endoscope, within a subject, displayed by an image display device;

FIG. 10A is a diagram illustrating a case where a template is set to a specified region within a given image;

FIG. 10B is a diagram illustrating a case where a template is disposed in a specified region within a given image for calculating a correlation level; and

FIG. 10C is a diagram illustrating an example of retrieving a template that is most similar to a template that is set within an image for calculating a correlation level.

### DETAILED DESCRIPTION OF THE PREFERRED EMBODIMENTS

Hereinafter, a receiving device and a capsule endoscope system according to an embodiment of the present invention will be described with reference to the drawings. In the following description, a capsule endoscope system that includes a capsule endoscope that is inserted into the body of a subject to capture an in-vivo image of the subject is illustrated as an example of the receiving device and the capsule endoscope system according to the present invention. However, the present invention is not limited to this embodiment.

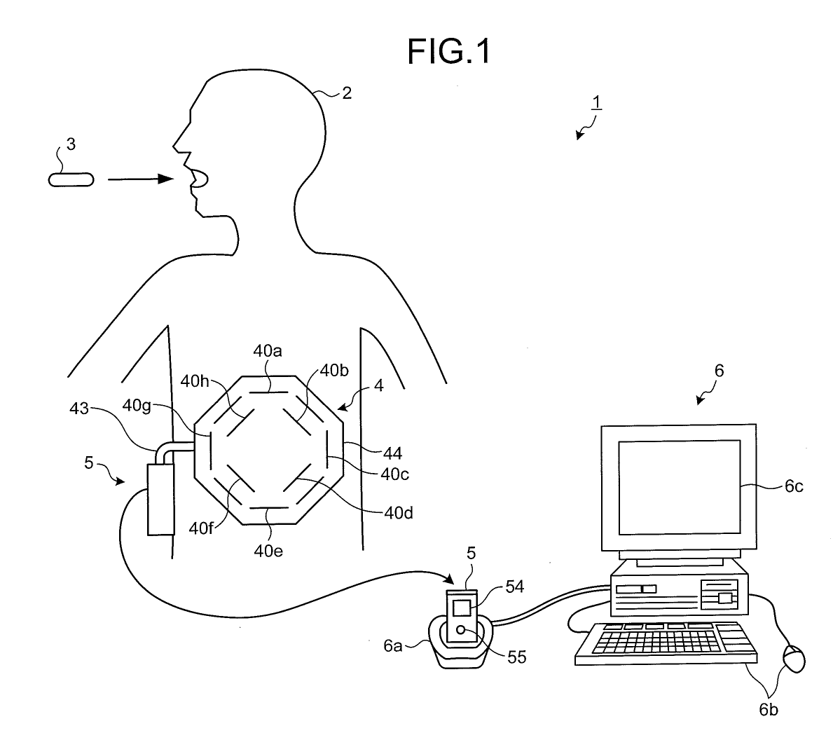

FIG. 1 is a schematic diagram illustrating an overall configuration of a capsule endoscope system 1 that uses a receiving device 5 according to a first embodiment of the present invention. As illustrated in FIG. 1, the capsule endoscope system 1 includes a capsule endoscope 3 that captures an in-vivo image of a subject 2, the receiving device 5 that receives a wireless signal wirelessly transmitted by the capsule endoscope 3 inserted into the subject 2 via a receiving antenna unit 4 and estimates the captured position of the in-vivo image data of the subject 2, captured by the capsule endoscope 3, and an image display device 6 that displays an image corresponding to the in-vivo image data of the subject 2, captured by the capsule endoscope 3.

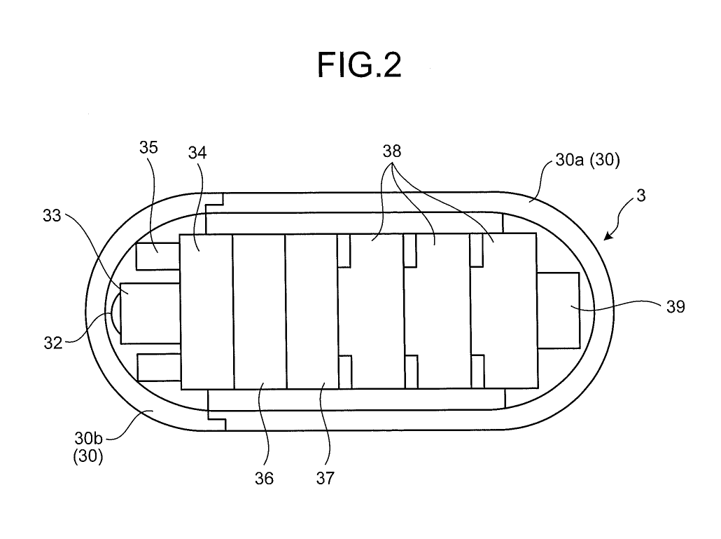

FIG. 2 is a cross-sectional view illustrating an overall internal configuration of the capsule endoscope 3. As illustrated in FIG. 2, the capsule endoscope 3 is accommodated in a capsule-shaped container (housing) 30 that is formed of an approximately cylindrical or oval hemispherical container 30a of which one end has a hemispherical dome shape and the other end is open and a hemispherical optical dome 30b that is fitted to the opening of the container 30a and liquid-tightly seals the container 30a. The capsule-shaped container 30 (30a, 30b) has such a size that it can be swallowed by the subject 2, for example. Moreover, in the first embodiment, at least the optical dome 30b is formed of a transparent material.

Moreover, the capsule endoscope 3 includes an objective lens 32 that focuses light entering through the optical dome 30b, a lens frame 33 to which the objective lens 32 is attached, an imaging unit 34 that converts the light having passed through the objective lens 32 into an electrical signal to form an imaging signal, an illuminating unit 35 that illuminates the inside of the subject 2 during imaging, a circuit board 36 that includes a processing circuit or the like that drives the imaging unit 34 and the illuminating unit 35, respectively and generates an image signal from the imaging signal input from the imaging unit 34, a transceiving circuit 37 that transmits the image signal and receives signals from the receiving device 5 or the like outside the body cavity, a plurality of button batteries 38 that supplies power to the respective functional units, and an antenna 39.

The capsule endoscope 3 passes through the throat inside the subject 2 by being swallowed into the subject 2 and moves along the body cavity according to a peristaltic motion of the lumen of the digestive tract. The capsule endoscope 3 sequentially captures the images of the body cavity of the subject 2 at very small time intervals (for example, every 0.5 seconds) while moving along the body cavity to generate the captured in-vivo image data of the subject 2 and sequentially transmits the image data to the receiving device 5. In the first embodiment, although a position estimating process may be performed based on the image signals of the image data captured by the imaging unit 34 of the capsule endoscope 3, it is preferable to generate transmission signals including the captured image signals and reception strength detection signals for detecting the position of the capsule endoscope 3 and to perform a position detecting process based on the reception strength detection signals from which reception strength can be easily detected.

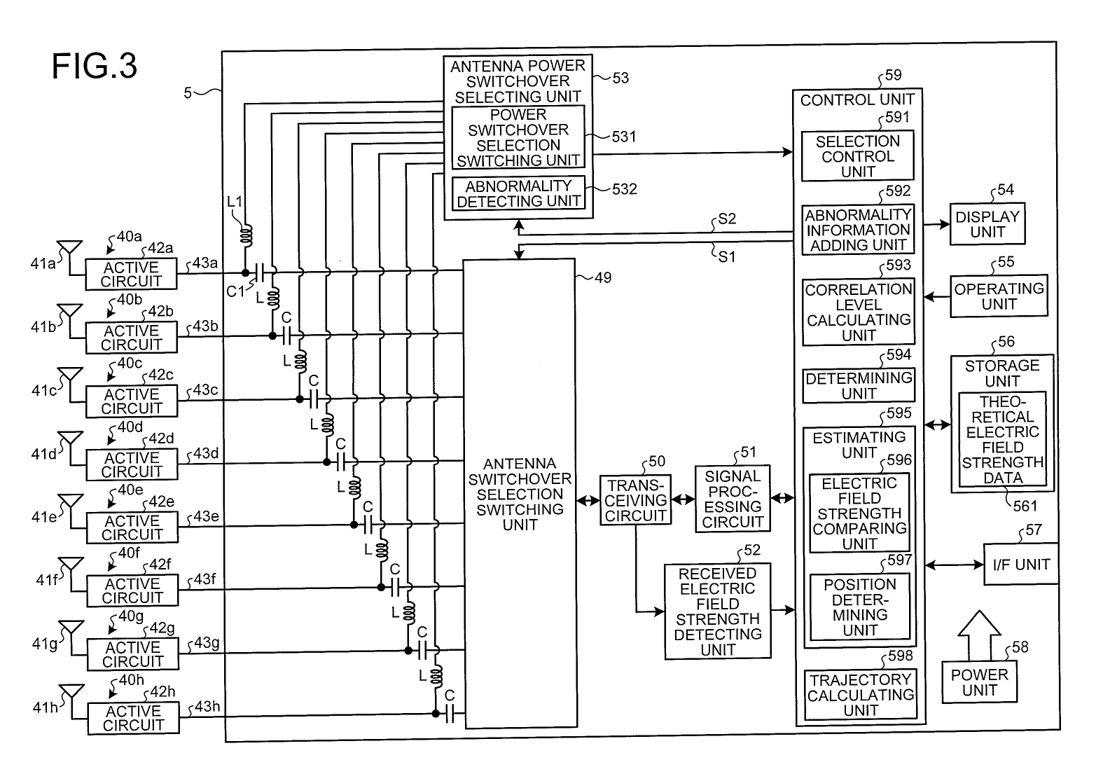

A position detecting apparatus includes a sheet-shaped receiving antenna unit 4 on which a plurality of receiving antennas 40 (40a, 40b, 40c, 40d, 40e, 40f, 40g, and 40h) is arranged, and the receiving device 5. The receiving device 5 is connected to the receiving antenna unit 4 by an antenna cable 43. The receiving device 5 receives a wireless signal transmitted from the capsule endoscope 3 via ach of the receiving antennas 40a to 40h. The receiving device 5 detects received electric field strength of the wireless signal 5 received from the capsule endoscope 3 with respect to each of the receiving antennas 40a to 40h and acquire the in-vivo image data of the subject 2 based on the received wireless signal. The receiving device 5 stores the received electric field strength information of the respective receiving antennas 40a to 40h, time information, and the like in a storage unit (see FIG. 3) in correlation with the received image data.

During the period when an image is captured by the capsule endoscope 3, the receiving device 5 is carried on the subject 2 until the capsule endoscope 3 is excreted from the subject 2 after being inserted through the mouth of the subject 2 and moving inside the digestive tract, for example. After the examination of the capsule endoscope 3 ends, the receiving device 5 is separated from the subject 2 and connected to the image display device 6 in order to transmit information such as the image data received from the capsule endoscope 3.

The receiving antennas 40a to 40h are disposed at specified positions of a sheet 44, for example, the positions corresponding to the internal organs of the subject 2, which are the passages of the capsule endoscope 3 when the receiving antenna unit 4 is attached to the subject 2. The arrangement of the receiving antenna 40a to 40h may be changed optionally according to the purposes of examinations or diagnoses. In this embodiment, although eight receiving antennas are used, the number of receiving antennas is not limited to 8 but may be larger or smaller than 8.

The image display device 6 is configured using a workstation or a personal computer that includes a monitor unit 6c such as a liquid crystal display. The image display device 6 displays an image corresponding to the in-vivo image data of the subject 2 acquired via the receiving device 5. The image display device 6 is connected to a cradle 6a that reads image data from the memory of the receiving device 5 and an operation input device 6b such as a keyboard or a mouse. When the cradle 6a includes the receiving device 5, the cradle 6a acquires image data from the memory of the receiving device 5 and related data such as received electric field strength information, time information, and identification information of the capsule endoscope 3 correlated with the image data and transmits various items of the acquired information to the image display device 6. The operation input device 6b receives the input of a user. The user operates the operation input device 6b to observe a biological region (for example, the throat, the stomach, the small intestine, the large intestine, and the like) inside the subject 2 while seeing the in-vivo images of the subject 2 sequentially displayed by the image display device 6 to thereby diagnose the subject 2.

Next, the configuration of the receiving device 5 illustrated in FIG. 1 will be described in detail. FIG. 3 is a block diagram illustrating the configuration of the receiving device 5 illustrated in FIG. 1.

As illustrated in FIG. 3, the receiving device 5 includes the respective receiving antennas 40a to 40h as described above, an antenna switchover selection switching unit 49 that selectively switches the receiving antennas 40a to 40h, a transceiving circuit 50 that performs processing such as demodulation on the wireless signal received via any one of the receiving antennas 40a to 40h selected by the antenna switchover selection switching unit 49, a signal processing circuit 51 that performs signal processing of extracting image data and the like from the wireless signal output from the transceiving circuit 50, a received electric field strength detecting unit 52 that detects a received electric field strength based on the strength of the wireless signal output from the transceiving circuit 50, an antenna power switchover selecting unit 53 that selectively switches the receiving antennas 40a to 40h to supply power to any one of the receiving antennas 40a to 40h, a display unit 54 that displays an image corresponding to the image data received from the capsule endoscope 3, an operating unit 55 that inputs operational instructions, a storage unit 56 that stores various items of information including the image data received from the capsule endoscope 3, an I/F unit 57 that transmits and receives data to and from the image display device 6 via the cradle 6a, a power unit 58 that supplies power to each unit of the receiving device 5, and a control unit 59 that controls the operation of the receiving device 5.

The receiving antenna 40a includes an antenna unit 41a, an active circuit 42a, and an antenna cable 43a. The antenna unit 41a is configured using an open antenna or a loop antenna, for example, and receives the wireless signal transmitted from the capsule endoscope 3. The active circuit 42a is connected to the antenna unit 41a so as to perform processing such as impedance matching of the antenna unit 41a and amplification, attenuation, and the like of the received wireless signal. The antenna cable 43a is configured using a coaxial cable and has one end electrically connected to the active circuit 42a and the other end electrically connected to the antenna switchover selection switching unit 49 and the antenna power switchover selecting unit 53 of the receiving device 5. The antenna cable 43a transmits the wireless signal received by the antenna unit 41a to the receiving device 5 and transmits power supplied from the receiving device 5 to the active circuit 42a. Since the receiving antennas 40b to 40h have the same configuration as the receiving antenna 40a, description thereof will not be provided.

The antenna switchover selection switching unit 49 is configured using a mechanical switch, a semiconductor switch, or the like. The antenna switchover selection switching unit 49 is electrically connected to the receiving antennas 40a to 40h via a capacitor C1. When a switchover signal S1 that switches the receiving antennas 40a to 40h that receive the wireless signal is input from the control unit 59, the antenna switchover selection switching unit 49 selects one of the receiving antennas 40 indicated by the switchover signal S1 and outputs the wireless signal received via the selected one of the receiving antennas 40a to 40h to the transceiving circuit 50. The capacitances of the capacitors connected to the receiving antennas 40a to 40h are the same as the capacitance of the capacitor C1.

The transceiving circuit 50 performs specified processing (for example, demodulation and amplification) on the wireless signal received via one of the receiving antennas 40 (40a to 40h) selected by the antenna switchover selection switching unit 49 and outputs the processed wireless signal to the signal processing circuit 51 and the received electric field strength detecting unit 52.

The signal processing circuit 51 extracts image data from the wireless signal input from the transceiving circuit 50, performs specified processing (for example, image processing and A/D conversion) on the extracted image data, and outputs the processed image data to the control unit 59.

The received electric field strength detecting unit 52 detects a received electric field strength corresponding to the strength of the wireless signal input from the transceiving circuit 50 and outputs a received signal strength indicator (RSSI) corresponding to the detected received electric field strength to the control unit 59.

The antenna power switchover selecting unit 53 is electrically connected to the receiving antennas 40a to 40h via a coil L1. The antenna power switchover selecting unit 53 supplies power to one of the receiving antennas 40a to 40h selected by the antenna switchover selection switching unit 49 via the antenna cable 43 (43a to 43h). The antenna power switchover selecting unit 53 includes a power switchover selection switching unit 531 and an abnormality detecting unit 532. The electrical characteristics of the coils connected to the receiving antennas 40a to 40h are the same as the electrical characteristics of the coil L1.

The power switchover selection switching unit 531 is configured using a mechanical switch, a semiconductor switch, or the like. When a select signal S2 that selects one of the receiving antennas 40a to 40h to which power is to be supplied is input from the control unit 59, the power switchover selection switching unit 531 selects one of the receiving antennas 40a to 40h indicated by the select signal S2 and supplies power to only the selected one of the receiving antennas 40a to 40h.

When an abnormality occurs in the receiving antennas 40a to 40h to which power is to be supplied, the abnormality detecting unit 532 outputs an abnormality signal to the control unit 59 to information that an abnormality has occurred in the receiving antennas 40a to 40h to which power is supplied.

The display unit 54 is configured using a display panel that is formed from liquid crystals, organic electro luminescence (EL), or the like The display unit 54 displays various items of information such as an image corresponding to the image data captured by the capsule endoscope 3, an operating state of the receiving device 5, patient information of the subject 2, and an examined date.

The operating unit 55 can input an instruction signal such as an instruction to change an imaging cycle of the capsule endoscope 3. When an instruction signal is input by the operating unit 55, the signal processing circuit 51 transmits the instruction signal to the transceiving circuit 50, and the transceiving circuit 50 modulates the instruction signal and transmits the modulated signal to the receiving antennas 40a to 40h. The signals transmitted from the receiving antennas 40a to 40h are received by the antenna 39 and demodulated by the transceiving circuit 37, and the circuit board 36 performs an operation or the like of changing the imaging cycle, for example, in response to the instruction signal.

The storage unit 56 is configured using a semiconductor memory such as a flash memory or a random access memory (RAM) that is fixedly provided inside the receiving device 5. The storage unit 56 has theoretical electric field strength data 561 that is used for estimating the position and direction of the capsule endoscope 3 in the subject 2, in which the image data was captured. The theoretical electric field strength data 561 is theoretical data of the received electric field strength of the wireless signal received by the receiving antennas 40a to 40h, according to the position and direction of the capsule endoscope 3 in the subject 2. Moreover, the storage unit 56 stores the image data captured by the capsule endoscope 3 and various items of information correlated with the image data (for example, the estimated position and direction information of the capsule endoscope 3, the received electric field strength, and the identification information for identifying the receiving antenna from which the wireless signal was received). Further, the storage unit 56 stores various programs executed by the receiving device 5. The storage unit 56 may have a recording medium interface function of storing information in a recording medium such as a memory card from the outside and reading information stored in the recording medium.

The I/F unit 57 has a communication interface function and transmits and receives data to and from the image display device 6 via the cradle 6a.

The power unit 58 is configured using a battery that is detachably attached to the receiving device 5 and a switch unit that is turned on and off. The power unit 58 supplies driving power necessary for each configuration unit of the receiving device 5 in its ON state and stops the driving power supplied to each configuration unit of the receiving device 5 in its OFF state.

The control unit 59 is configured using a central processing unit (CPU) or the like. The control unit 59 reads and executes programs from the storage unit 56 and transmits instructions, data, and the like to each configuration unit of the receiving device 5 to thereby control the operation of the receiving device 5 in a centralized manner. The control unit 59 includes a selection control unit 591, an abnormality information adding unit 592, a correlation level calculating unit 593, a determining unit 594, an estimating unit 595, and a trajectory calculating unit 598.

The selection control unit 591 performs control of selecting one of the receiving antennas 40a to 40h that receive the wireless signal transmitted from the capsule endoscope 3 and supplying power to only the selected one of the receiving antennas 40a to 40h. Specifically, the selection control unit 591 performs control of selecting one receiving antenna 40 that receives the wireless signal transmitted from the capsule endoscope 3 based on the received electric field strengths of the receiving antennas 40a to 40h detected by the received electric field strength detecting unit 52 and supplying power to only the selected one of the receiving antennas 40a to 40h. The selection control unit 591 drives the antenna switchover selection switching unit 49 every specified points in time (for example, every 100 msec) to sequentially select the receiving antennas 40a to 40h that receive the wireless signal among the receiving antennas 40a to 40h to allow the received electric field strength detecting unit 52 to detect the received electric field strength.

When the abnormality detecting unit 532 detects an abnormality in any one of the receiving antennas 40a to 40h, the abnormality information adding unit 592 adds abnormality information to the wireless signal received by the receiving antenna 40 to indicate that an abnormality has occurred in any one of the receiving antennas 40a to 40h and outputs the wireless signal to the storage unit 56. Specifically, the abnormality information adding unit 592 adds abnormality information (flag) to the image data, which is obtained by the signal processing circuit 51 performing signal processing on the wireless signal received by the receiving antennas 40a to 40h, and outputs the image data to the storage unit 56.

The correlation level calculating unit 593 calculates a correlation level between each of the images received by the receiving antennas 40a to 40h and the previously received image.

The determining unit 594 determines whether the position and/or direction of the capsule endoscope 3 in the subject 2 is changed based on the correlation level between the images calculated by the correlation level calculating unit 593. When the determining unit 594 determined that the position or the direction of the image is changed, the estimating unit 595 to be described below performs a process of estimating the position and direction of the image.

The estimating unit 595 includes an electric field strength comparing unit 596 and a position determining unit 597. The electric field strength comparing unit 596 calculates a residual sum of squares between the received electric field strengths of the wireless signal received by the receiving antennas 40a to 40h and the theoretical electric field strengths stored in the storage unit 56 for each possible position and direction of the capsule endoscope 3 in the subject 2.

The position determining unit 597 determines the position and direction of the capsule endoscope 3 in which the image data was captured, based on the residual sum of squares calculated by the electric field strength comparing unit 596 or the sum of absolute residuals. The position determining unit 597 determines the region and direction in which the residual sum of squares is smallest as the position and direction of the capsule endoscope 3 in which the image data was captured.

The trajectory calculating unit 598 calculates the moving trajectory of the capsule endoscope 3 in the subject 2 based on the position information of the capsule endoscope 3, determined by the position determining unit 597 for each image data.

In the first embodiment, the receiving device 5 includes the correlation level calculating unit 593 that calculates a correlation level between an image and a previously received image, the determining unit 594 that determines whether the position and/or direction of the capsule endoscope 3 is changed based on the correlation level calculated by the correlation level calculating unit 593, and the estimating unit 595 that performs a position detecting process. The estimating unit 595 performs a process of estimating the position and direction of an image in which the determining unit 594 determines that the position and direction of the capsule endoscope 3 is changed. Hereinafter, the process in the receiving device 5 of the first embodiment, of estimating the position and direction of the capsule endoscope 3 will be described in detail.

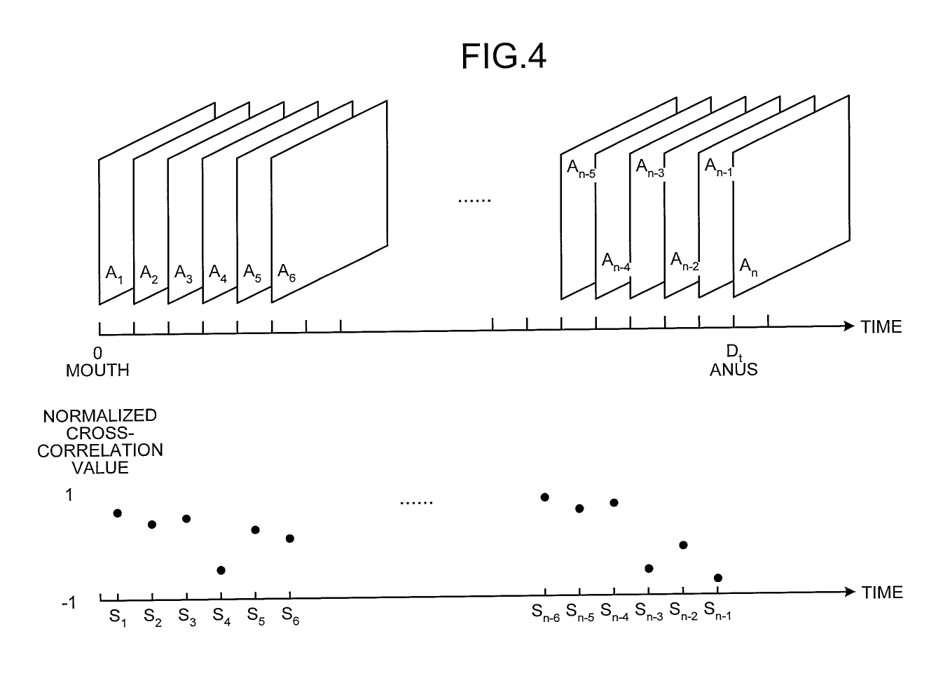

In the first embodiment, first, the correlation level calculating unit 593 calculates a correlation level between the image captured by the capsule endoscope 3 and a previously captured image. FIG. 4 is a diagram illustrating the relation between the images captured by the capsule endoscope 3 and the correlation levels.

As illustrated in FIG. 4, when images A₁ to Aₙ are captured until the capsule endoscope 3 is excreted from the anus after it is inserted through the mouth of the subject 2, the correlation level calculating unit 593 calculates correlation levels S₁ to Sₙ₋₁(Sₙ₋₁ is a correlation level between “Aₙ” and “Aₙ₋₁”) between all images A₂ to Aₙ except the image A₁ captured first and the previously captured images.

For example, when calculating the correlation level S₁, regions H₂(i, j) and H₁(i, j) (where i=1, 2, . . . , p; j=1, 2, . . . , q) are set for pixels that belong to the images A₂ and A₁. Moreover, a normalized cross-correlation value is calculated from Expressions (1) and (2) below based on the pixel values of the regions H₂(i, j) and H₁(i, j).

$$\begin{matrix}{S}_{( m - 1 )} = \frac{{\sum }_{j = 1}^{q} {\sum }_{i = 1}^{p} \{ {H}_{( m - 1 )} ( i , j ) - {μ}_{( m - 1 )} \} \{ {H}_{m} ( i , j ) - {μ}_{m} \}}{\sqrt{{\sum }_{j = 1}^{q} {\sum }_{i = 1}^{p} {\{ {H}_{( m - 1 )} ( i , j ) - {μ}_{( m - 1 )} \}}^{2} {\{ {H}_{m} ( i , j ) - {μ}_{m} \}}^{2}}} \\ ( m = 2 , 3 , … , n ) & ( 1 ) \\ {μ}_{m} = \frac{1}{pq} {\sum }_{j = 1}^{q} {\sum }_{i = 1}^{p} {H}_{m} ( i , j ) \\ ( m = 2 , 3 , … , n ) & ( 2 )\end{matrix}$$

The normalized cross-correlation value has a value of −1 to 1, and the closer it is to “1”, the higher is the similarity between images. Thus, if the normalized cross-correlation value between two images sequentially captured at different points in time is larger than a specified threshold value, it is determined that no scene change has occurred (the position or the direction of the capsule endoscope 3 is not changed). Moreover, if the normalized cross-correlation value is smaller than the specified threshold value, it is determined that a scene change has occurred (the position or the direction of the capsule endoscope 3 is changed).

In FIG. 4, since the values of the correlation levels S₄, Sₙ₋₃, Sₙ₋₂, and Sₙ₋₁ are close to “−1”, it can be determined that a scene change has occurred. Thus, the position and direction of the capsule endoscope 3 are estimated for the images A₅, Aₙ₋₂, Aₙ₋₁, and Aₙ. The normalized cross-correlation value may be calculated for a specific region at the center of an image rather than the entire image to determine the correlation level. Moreover, an image may be divided into a plurality of regions, and the normalized cross-correlation value may be calculated for each of the divided regions to determine the movement (a change in the position and/or direction of the capsule endoscope 3) of the capsule endoscope 3.

Further, the correlation level may be determined according to the residual sum of squares between pixel values instead of the normalized cross-correlation value. When calculating the residual sum of squares, regions H₂(i, j) and H₁(i, j) (where i=1, 2, . . . , p; j=1, 2, . . . , q) are set for pixels that belong to the images A₂ and A₁. Then, the residual sum of squares is calculated from Expression (3) below based on the pixel values of the regions H₁(i, j) and H₂(i, j).

$${S}_{( m - 1 )} = {\sum }_{j = 1}^{q} {\sum }_{i = 1}^{p} {\{ {H}_{m} ( i , j ) - {H}_{( m - 1 )} ( i , j ) \}}^{2} ( m = 2 , 3 , … , n ) \qquad ( 3 )$$

The closer the residual sum of squares is to “0”, the higher is the similarity between images. Thus, it can be determined that the closer the residual sum of squares is to “0”, the more two images are similar to each other. Therefore, if the residual sum of squares is smaller than a specified threshold value, it is determined that no scene change has occurred (the position or the direction of the capsule endoscope 3 is not changed). Moreover, if the residual sum of squares is larger than the specified threshold value, it is determined that a scene change has occurred (the position or the direction of the capsule endoscope 3 is changed).

Further, since each image has pixel values for the respective color components of R (red), G (green), and B (blue), the normalized cross-correlation value and the residual sum of squares can be also calculated for the respective color components. The correlation level between images can be also determined from the mean value of the normalized cross-correlation values or the residual sums of squares of the respective color components.

Hereinafter, a method of calculating the position and direction of the capsule endoscope 3 when the determining unit 594 determines that the position and the direction of the capsule endoscope 3 are changed will be described.

In the first embodiment, the electric field strength comparing unit 596 calculates the residual sum of squares between the received electric field strengths of the wireless signal received by the receiving antennas 40a to 40h and the theoretical electric field strengths stored in the storage unit 56 with respect to each possible position and direction of the capsule endoscope 3 in the subject 2. The position determining unit 597 determines the region and direction, in which the residual sum of squares calculated by the electric field strength comparing unit 596 is smallest, as the position and the direction of the capsule endoscope 3, in which the image data was captured.

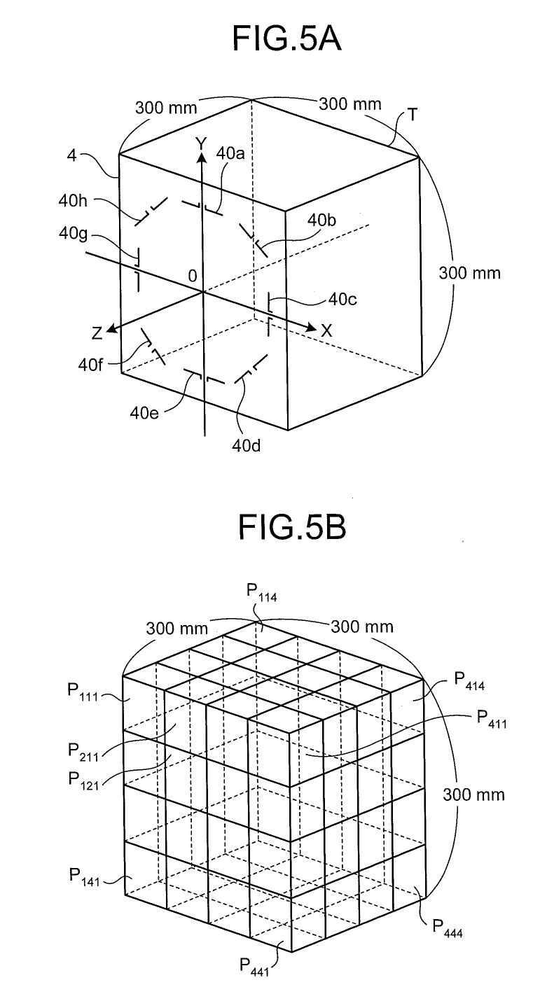

Here, a method of calculating the theoretical electric field strength data 561 stored in advance in the storage unit 56 will be described. First, a specified possible presence region T where the capsule endoscope 3 is possibly present is set within the subject 2 in which the capsule endoscope 3 is inserted depending on the purposes of examinations or diagnoses. The possible presence region T is a region that is set according to the body size of the subject 2 and is a cubic region of 300 mm×300 mm×300 mm as illustrated in FIG. 5A, for example. The possible presence region T is set so that the sheet-shaped surface of the receiving antenna unit 4 is identical to one boundary surface of the possible presence region T. In the case of FIG. 5A, the receiving antenna unit 4 is provided on the XY plane which is one boundary surface of the possible presence region T.

The possible presence region of the capsule endoscope 3 is divided into a plurality of partial regions depending on desired accuracy. FIG. 5B illustrates a case where the possible presence region T is divided in quarters in the respective axis directions of an orthogonal coordinate system XYZ of which the origin is at the center of the boundary surface on which the receiving antenna unit 4 is located and of which the three orthogonal axes (X, Y, and Z-axes) are parallel to sides of the possible presence region T. In this case, the possible presence region T is divided into 64 (=4×4×4) partial regions. The partial regions are labeled with P₁₁₁, P₁₁₂, P₁₁₃, P₁₁₄, P₁₂₁, P₁₂₂, . . . , P₁₄₄, P₂₁₁, P₂₁₂, . . . , and P₄₄₄. When the capsule endoscope 3 is present in a partial region Pᵢⱼₖ, it is assumed that the capsule endoscope 3 is located at the center G_{xyz} of the partial region Pᵢⱼₖ.

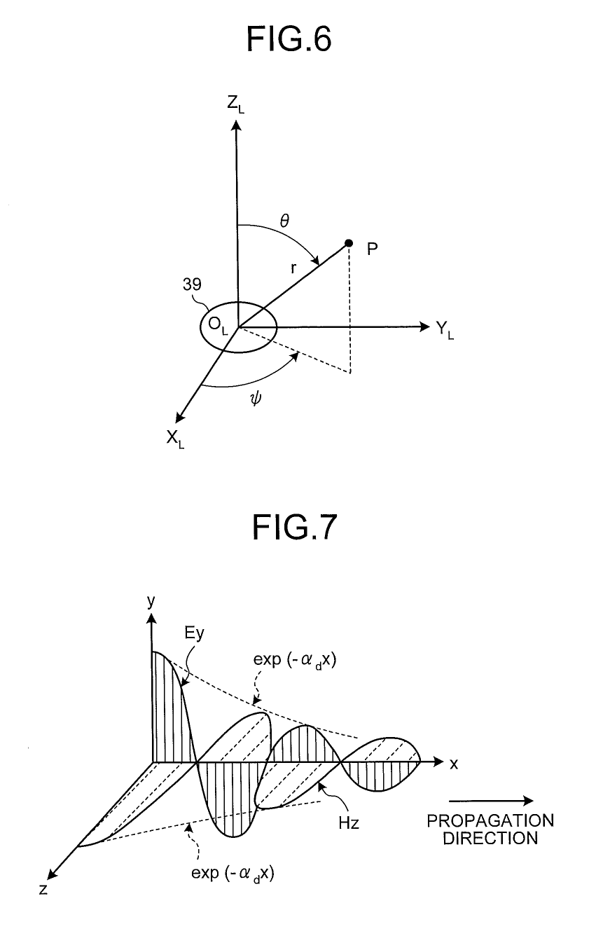

In the following description, as illustrated in FIG. 6, an orthogonal coordinate system X_{L}Y_{L}Z_{L} of which the origin (O_{L}) is at the center of the circular loop-shaped antenna 39 disposed in the capsule endoscope 3, and the Z_{L} axis is the direction normal to an aperture surface of the circular loop. In this orthogonal coordinate system X_{L}Y_{L}Z_{L}, polar coordinate components of an electromagnetic field that a current flowing in the antenna 39 forms at an arbitrary position P are given by Expression (4) below.

Hᵣ=(IS/2π)(jk/r²+1/r³)exp(−jkr)cos θ

H_{θ}=(IS/470(−k²/r+jk/r²+1/r³)exp(−jkr)sin 74

E_{ψ}=−(jωμIS/4π)(jk/r+1/r²)exp(−jkr)sin θ (4)

Here, “Hᵣ” and “H_{θ}” denote magnetic field components and “E_{ψ}” denotes an electric field component. Moreover, “I” denotes the current flowing in the antenna 39 and “S” denotes the area of the aperture surface of the circular loop that forms the antenna 39. Further, k=ω(εμ)^{1/2} (where “ε” is the electric constant and “μ” is the permeability) is the wave number, and “j” is the imaginary unit. Here, in Expression (4), the r⁻¹ term is a radiation electromagnetic field component, the r⁻² term is an induction electromagnetic field component, and the r⁻³ term is an electrostatic magnetic field component.

When the frequency of an electromagnetic field generated by the antenna 39 that is disposed in the capsule endoscope 3 is high and the distance between the capsule endoscope 3 and the receiving antennas 40 (40a to 40h) attached to the body surface of the subject 2 is sufficiently large as illustrated in FIG. 1, the radiation electromagnetic field component of the electromagnetic field (electromagnetic wave) that reaches the receiving antennas 40 (40a to 40h) is the largest. Thus, the electrostatic magnetic field component and the induction electromagnetic field component are smaller than the radiation electromagnetic field component and can be negligible. Thus, Expression (4) can be rewritten to Expression (5) below.

Hᵣ=0

H_{θ}=(IS/470(−k²/r)exp(−jkr)sin θ

E_{ψ}=−(jωμIS/470(jk/r)exp(−jkr)sin θ (5)

If the receiving antenna 40 attached to the body surface of the subject 2 is an electric field detection antenna for detecting an electric field, the equation necessary for detecting the same is the electric field E_{ψ} in Expression (5). Thus, the instantaneous value of the electric field E_{ψ} is obtained by multiplying both sides of the electric field E_{ψ} of Expression (5) with “exp(jωt)” using the theory of alternating current and extracting the real part.

E_{ψ}exp(jωt)

=−(jωμIS/4π)(jk/r)exp(−jkr)sin θexp(jωt)

=(ωμISk/4πr)(cos U+j sin U)sin θ (6)

where U=ωt−kr

Here, when the real part of Expression (6) is extracted, the instantaneous value E′_{ψ} of the electric field is expressed as follows.

E′_{ψ}=(ωμISk/4πr)cos U sin θ (7)

Moreover, when Expression (7) is expressed by the orthogonal coordinate system X_{L}Y_{L}Z_{L}, the components E_{Lx}, E_{Ly}, E_{Lz} are obtained as follows.

E_{Lx}=E′_{ψ} sin ψ=(ωμISk/4πr²)cos U·(−y_{L})

E_{Ly}=E′_{ψ} cos ψ=(ωμISk/4πr²)cos U·x_{L}

E_{Lz}=0 (8)

When an electromagnetic wave propagates in a medium, as illustrated in FIG. 7, the energy of the electromagnetic wave is absorbed by the medium in which the electromagnetic wave propagates due to the characteristics (electrical conductivity or the like) of the medium. An electromagnetic wave is exponentially attenuated by an attenuation factor “α_{d}” as it propagates in the x-direction, for example, which can be expressed as Expression (9) below.

Aᵣ=exp (−α_{d}x)

α_{d}=(ω²εμ/2)^{1/2}[(1+κ²/(ω²ε²))^{1/2}−1]^{1/2} (9)

where ε=εₒεᵣ (εₒ: electric constant of vacuum, εᵣ: the relative electric constant), μ=μₒμᵣ (μₒ: the permeability of vacuum, the relative permeability), ω is the angular frequency, and κ is the electric conductivity.

Thus, when the in-vivo characteristics are taken into consideration, the respective components E_{Lx}, E_{Ly}, and E_{Lz} of the orthogonal coordinate system X_{L}Y_{L}Z_{L} of the instantaneous value E_{L} of the electric field are expressed as Expression (10) below.

E_{Lx}=AᵣE′_{ψ} sin ψ=exp(−α_{d}r)(ωμISk/4πr²)cos U·(−y_{L})

E_{Ly}=AᵣE′_{ψ} cos ψ=exp(−α_{d}r)(ωμISk/4πr²)cos U·x_{L}

E_{Lz}=0 (10)

Further, an equation that transforms a position P(X_{L}, Y_{L}, Z_{L}) in the coordinate system X_{L}Y_{L}Z_{L} based on the antenna 39 of the capsule endoscope 3 into a corresponding position in a coordinate system X_{W}Y_{W}Z_{W} of which the origin is at the center (“O” in FIG. 5A) of the receiving antenna unit 4 that is attached to the subject 2 is as follows.

$$\begin{matrix}( \begin{matrix}{x}_{LP} \\ {y}_{LP} \\ {z}_{LP}\end{matrix} ) = {R}^{- 1} [ ( \begin{matrix}{x}_{WP} \\ {y}_{WP} \\ {z}_{WP}\end{matrix} ) - ( \begin{matrix}{x}_{WG} \\ {y}_{WG} \\ {z}_{WG}\end{matrix} ) ] \\ = ( \begin{matrix}{R}_{00} & {R}_{01} & {R}_{02} \\ {R}_{10} & {R}_{11} & {R}_{12} \\ {R}_{20} & {R}_{21} & {R}_{22}\end{matrix} ) [ ( \begin{matrix}{x}_{WP} \\ {y}_{WP} \\ {z}_{WP}\end{matrix} ) - ( \begin{matrix}{x}_{WG} \\ {y}_{WG} \\ {z}_{WG}\end{matrix} ) ]\end{matrix} \qquad ( 11 )$$

Here, (x_{WP}, y_{WP}, z_{WP}) and (x_{wG}, y_{WG}, z_{WG}) denote the position P and the position G of the antenna 39, respectively, in the coordinate system X_{W}Y_{W}Z_{W}. Moreover, “R” on the right side of Expression (12) denotes a rotational matrix between the coordinate system X_{W}Y_{W}Z_{W} and the coordinate system X_{L}Y_{L}Z_{L} and is obtained by the following equation.

$$( \begin{matrix}{R}_{00} & {R}_{10} & {R}_{20} \\ {R}_{01} & {R}_{11} & {R}_{21} \\ {R}_{02} & {R}_{12} & {R}_{22}\end{matrix} ) = ( \begin{matrix}cos α cos β & - sin α & cos α sin β \\ sin α cos β & cos α & sin α sin β \\ - sin β & 0 & cos β\end{matrix} ) \qquad ( 12 )$$

where “α” is a rotational angle around the Z-axis and “β” is a rotational angle around the Y-axis.

Thus, the electric field E_{W} at an optional position P(x_{WP}, y_{WP}, z_{WP}) of the coordinate system X_{W}Y_{W}Z_{W} of which the origin is at the center (“O” in FIG. 5A) of the receiving antenna unit 4 that is attached to the subject 2 is expressed as follows.

$$Ew = ( \begin{matrix}{E}_{Wx} \\ {E}_{Wy} \\ {E}_{Wz}\end{matrix} ) = R ( \begin{matrix}{E}_{Lx} \\ {E}_{Ly} \\ {E}_{Lz}\end{matrix} ) = ( \begin{matrix}{R}_{00} & {R}_{10} & {R}_{20} \\ {R}_{01} & {R}_{11} & {R}_{21} \\ {R}_{02} & {R}_{12} & {R}_{22}\end{matrix} ) ( \begin{matrix}{E}_{Lx} \\ {E}_{Ly} \\ {E}_{Lz}\end{matrix} ) \qquad ( 13 )$$

When Expressions (10) to (12) are substituted into Expression (13), the equation of the electric field E_{W} is obtained as Expression (14) below.

$$( \begin{matrix}{E}_{Wx} \\ {E}_{Wy} \\ {E}_{Wz}\end{matrix} ) = \frac{{k}_{1}}{{r}^{2}} {e}^{- {α}_{d} r} ( \begin{matrix}0 & ( {z}_{WP} - {z}_{WG} ) & - ( {y}_{WP} - {y}_{WG} ) \\ - ( {z}_{WP} - {z}_{WG} ) & 0 & ( {x}_{WP} - {x}_{WG} ) \\ ( {y}_{WP} - {y}_{WG} ) & - ( {x}_{WP} - {x}_{WG} ) & 0\end{matrix} ) ( \begin{matrix}{g}_{x} \\ {g}_{y} \\ {g}_{z}\end{matrix} ) \qquad ( 14 )$$

where “k₁” is a constant, the vector (gₓ, g_{y}, g_{z}) denotes the direction “g” of the antenna 39. In the first embodiment, the direction (gₓ, g_{y}, g_{z}) of the antenna 39 is set in advance together with the position of the capsule endoscope 3, and the theoretical electric field strengths of the receiving antennas 40 when the capsule endoscope 3 is located at a specified region and faces a specified direction are calculated. The direction of the antenna 39 may be set by notching at an angle of 1 degree from the horizontal and vertical axes, for example, according to desired accuracy.

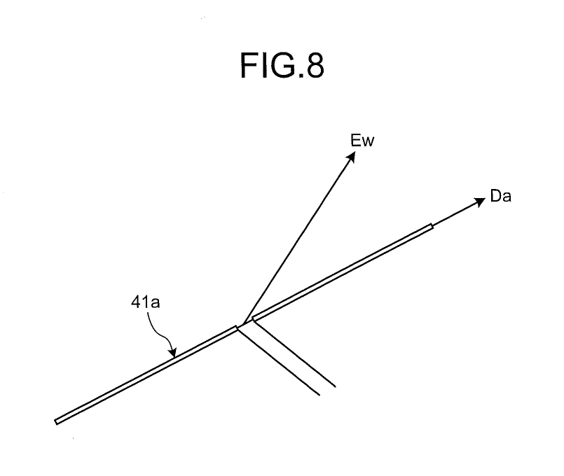

Moreover, electromotive force Vₜₐ detected when the electric field E_{W} generated by the antenna 39 is received by the receiving antenna 40a that constitutes the receiving antenna unit 4 can be calculated by the following equation using the inner product between the electric field E_{W} and a vector Dₐ=(Dₓₐ, D_{ya}, D_{za}) (see FIG. 8) that represents the direction of the receiving antenna 40a (the antenna unit 41a) of the receiving antenna unit 4 in a coordinate system based on the subject 2.

Vₜₐ=k₂(E_{Wx}Dₓₐ+E_{Wy}D_{ya}+E_{Wz}D_{za}) (15)

where k₂ is a constant. Similarly, the electromotive force V_{tb}, . . . , Vₜₕ when the electric field is received by the plurality of receiving antennas 40b to 40h of the receiving antenna unit 4 arranged on the body of the subject 2 is obtained.

In this manner, the theoretical electric field strengths Vₜᵢ received by the receiving antennas 40 are calculated and are stored in the storage unit 56 for each central position G of the divided region as the theoretical electric field strength data 561.

The electric field strength comparing unit 596 calculates the residual sum of squares between the received electric field strengths of the receiving antennas 40 and the theoretical electric field strengths stored in the storage unit 56 as the theoretical electric field strength data 561 calculated in this manner with respect to the central position G of each possible presence region of the capsule endoscope 3 and the direction “g” of the antenna 39. When the received electric field strength of the receiving antenna 40 is Vₘᵢ (i is a receiving antenna number, and i=a to h in this embodiment), the residual sum of squares “S” can be calculated by the following equation.

$$\begin{matrix}S = {\sum }_{i = a}^{h} {( {V}_{ti} - {V}_{mi} )}^{2} \\ = {( {V}_{ta} - {V}_{ma} )}^{2} + … + {( {V}_{th} - {V}_{mh} )}^{2}\end{matrix} \qquad ( 16 )$$

The electric field strength comparing unit 596 calculates the residual sum of squares between the received electric field strengths Vₘᵢ received by the receiving antennas 40 and the theoretical electric field strengths Vₜᵢ stored in the storage unit as the theoretical electric field strength data 561 calculated in this manner with respect to the central position G of each possible presence region of the capsule endoscope 3 and the direction “g” of the antenna 39. Thus, for example, by using the same number of CPUs (or a fractional number of the total number of central positions G to be estimated, or a number equal to or smaller than the fractional number) as the total number of central positions G to be estimated as the electric field strength comparing unit 596 in the estimating process at the same time, it is possible to accelerate the process of estimating the position and direction of the capsule endoscope 3.

The position determining unit 597 determines the central position G of the capsule endoscope 3 and the direction “g” of the antenna 39, in which the residual sum of squares “S” calculated by the electric field strength comparing unit 596 is the smallest, as the position and direction of the capsule endoscope 3.

Although the estimating unit 595 can estimate the position and direction of the capsule endoscope 3 in the subject 2 in this manner, besides this method, the position and direction of the capsule endoscope 3 may be obtained by iterative refinement using the Gauss-Newton method as disclosed in Japanese Laid-open Patent Publication No. 2007-283001, for example.

In this manner, the information on the estimated position and direction of the capsule endoscope 3 is stored sequentially (with time) in the storage unit 56 of the receiving device 5 together the image data and the frame number Nf of each image data. The trajectory calculating unit 598 estimates (calculates) the moving trajectory of the capsule endoscope 3 in the subject 2 from the sequentially stored position of the capsule endoscope 3.

The moving trajectory calculated in this manner is displayed on the image display device 6. Specifically, as illustrated in FIG. 1, when the receiving device 5 is connected to the cradle 6a, the image data and the frame number Nf and the information on the position and direction of the capsule endoscope 3 stored in the storage unit 56 from the receiving device 5 are transmitted to the image display device 6, and these items of information can be displayed on the monitor unit 6c.

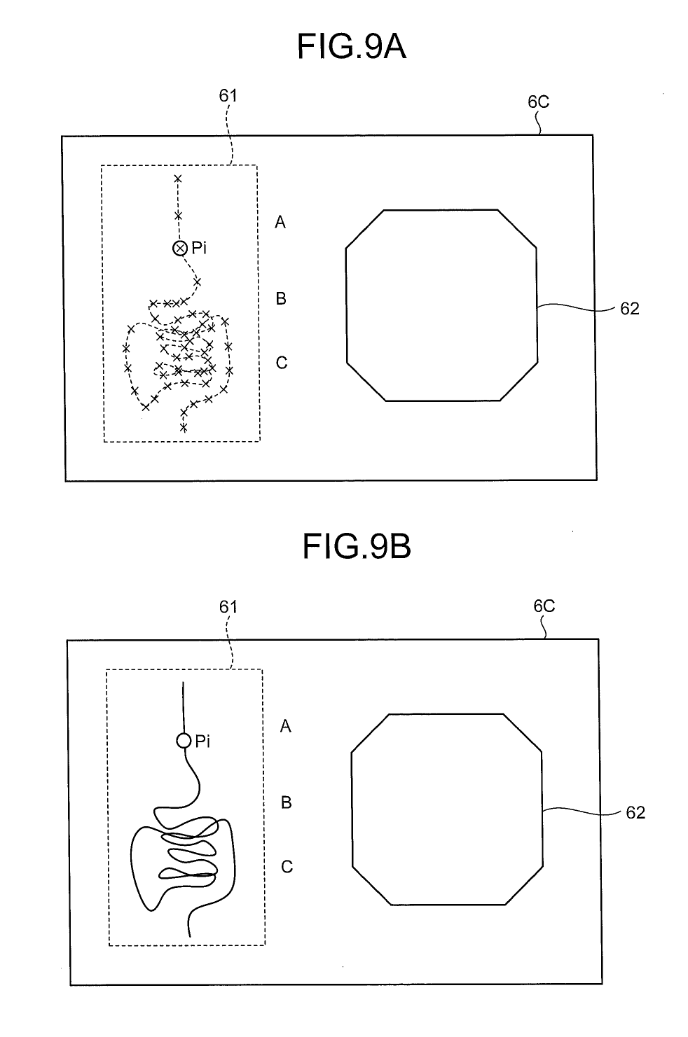

FIGS. 9A and 9B illustrate examples of the moving trajectory of the capsule endoscope 3 in the subject 2, displayed on the monitor unit 6c. As illustrated in FIG. 9A, the monitor unit 6c includes an auxiliary image region 61 that indicates the moving trajectory of the capsule endoscope 3 in the subject 2 by connecting the captured positions of the capsule endoscope 3 in the subject 2 by straight lines and a main image display region 62 that displays the image data captured by the capsule endoscope 3.

The symbols A, B, and C marked at the right side of the auxiliary image region 61 indicate approximate positions of organs in the body cavity, and specifically, the symbols A, B, and C indicate the throat, the small intestine, and the large intestine, respectively. Moreover, the position “Pᵢ” indicates the estimated captured position of the image data displayed in the main image display region 62. In addition to FIG. 9A in which the estimated captured position “Pᵢ” are connected by straight lines to exhibit these straight lines as a trajectory, as illustrated in FIG. 9B, for example, an interpolation process such as spline interpolation may be performed on adjacent captured positions, and the estimated captured positions of the capsule endoscope 3 may be displayed by connecting the same by a smooth curve.

As described above, since the possible presence region of the capsule endoscope 3 is divided into a plurality of small regions and the theoretical electric field strength Vₜᵢ according to the direction of the capsule endoscope 3 is stored in advance for each divided region, it is possible to reduce the processing load of calculating the theoretical electric field strength Vₜᵢ. Moreover, since the position and direction of the capsule endoscope 3 in which the image data was captured are determined based on numerical values obtained by a simple computation process of the residual sum of squares between the stored theoretical electric field strength Vₜᵢ and the received electric field strength Vₘᵢ actually received by the receiving antennas 40, it is possible to accelerate the position estimating process.

Further, since the sheet-shaped receiving antenna unit 4 on which the plurality of receiving antennas 40 are arranged is used, it is not necessary to adjust the arrangement positions of the respective receiving antennas 40 whenever examinations are performed. Further, since the receiving antenna unit 4 in which the arrangement positions of the receiving antennas 40 are determined in advance is used, it is possible to prevent deterioration of accuracy in the process of estimating the position and direction of the capsule endoscope 3 resulting from a shift in the arrangement of the respective receiving antennas 40.

In the first embodiment, the correlation level calculating unit 593 calculates the correlation level between images captured at different points in time, and the determining unit 594 determines a change in the position and direction of the capsule endoscope 3 in the subject 2 based on the calculated correlation level. The position and direction of the capsule endoscope 3 are estimated for only the image data in which the position and direction of the capsule endoscope 3 is determined to be changed. Thus, it is possible to shorten the time required for the position estimating process of the capsule endoscope 3 and to estimate the accurate moving trajectory of the capsule endoscope 3 in the subject 2.

In the first embodiment, the receiving device that estimates the position and direction of the capsule endoscope 3 has been described. However, the receiving device may estimate only one of the position and direction of the capsule endoscope 3. Moreover, in the first embodiment, the receiving device 5 includes the correlation level calculating unit 593, the determining unit 594, the estimating unit 595, and the trajectory calculating unit 598, and the receiving device 5 determines whether or not to estimate the position and direction during capturing, of the capsule endoscope 3 with respect to the captured image data to estimate the position and calculate the trajectory. However, the image display device 6 of the capsule endoscope system 1 may include the correlation level calculating unit, the determining unit, the estimating unit, and the trajectory calculating unit and may receive the image data transmitted from the receiving device to estimate the position and direction of the capsule endoscope in which the image data was captured.

In the first embodiment, the correlation level between images is determined based on the normalized cross-correlation of the pixel values of an entire image. However, in a second embodiment, a specified region of an image is extracted as a template, a region that is most similar to the specified region among the other image regions in which the correlation level is calculated is retrieved by a block matching method, and a change in the position and direction of the capsule endoscope is detected from the movement amount of the retrieved region.

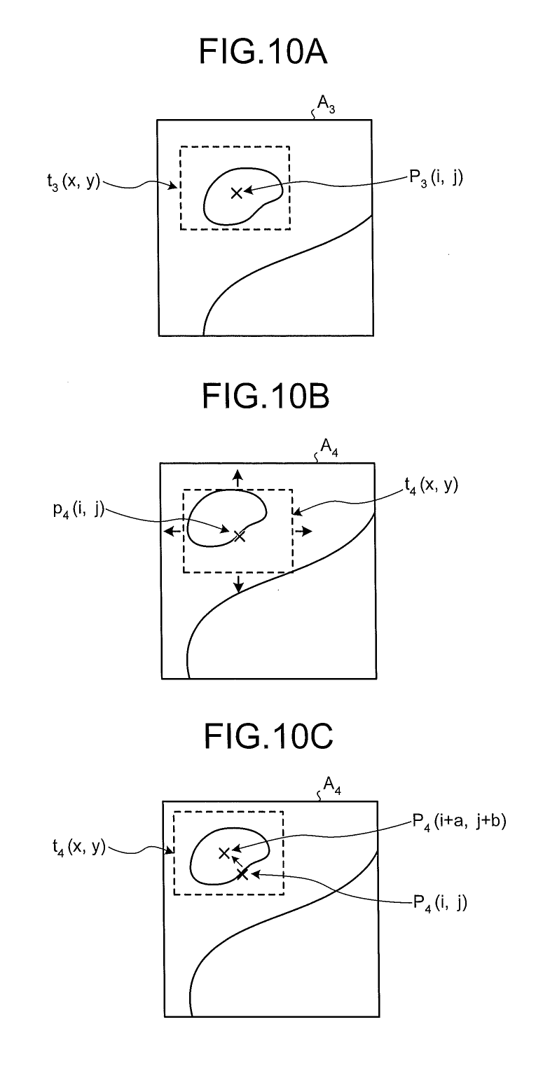

As an example, a case of calculating the correlation level between images A₃ and A₄ by the block matching method will be described. FIG. 10A is a diagram illustrating a case where a template is set to a specified region in the image A₃. FIG. 10B is a diagram illustrating a case where the corresponding region is set in the image A₄. FIG. 10C is a diagram illustrating an example of retrieving a region that is most similar to the template of the image A₃ from the image A₄.

As illustrated in FIG. 10A, a template t₃(x, y) that represents a detection target is set in a specified region in the image A₃ so that the center overlaps a point P₃(i, j) in the image A₃. Similarly, as illustrated in FIG. 10B, a region t₄(x, y) having the same size and position as the template t₃(x, y) is set at a point P₄(i, j) in the image A₄.

The correlation level calculating unit 593 calculates a correlation level S₃(i, j) between the template t₃(x, y) and the region t₄(x, y). Here, the correlation level S₃(i, j) is a normalized cross-correlation and is one that is obtained when m=4 on the right side of Expression (1).

Subsequently, the correlation level calculating unit 593 sets the region t₄(x, y) having the same size and position as the template t₃(x, y) at the point P₄(i+1, j) that is moved by “1” in the x-axis direction from the point P₄(i, j) in the image A₄ and calculates a correlation level S₃(i+1, j).

Similarly, the point P₄(i, j) is moved in the x-axis and/or y-axis directions within such a range that the region t₄(x, y) is within the image A₄, and a correlation level S₃(i+a, j+b) between the region t₄(x, y) around the moved point P₄(i+a, j+b) and the template t₃(x, y) is calculated (where a, b=1, 2, 3, . . . ). Here, S₃(i+a, j+b) is one that is obtained when i→i+a and j→j+b on the right side of Expression (1) and m=4.

The correlation level calculating unit 593 determines the region t₄(x, y) around the point P₄(i+a₀, j+b₀) at which the correlation level S₃(i+a, j+b) is the largest as a region that is most similar to the template t₃(x, y), and the movement amount M of the capsule endoscope 3 is calculated by the following equation.

M={square root over ((i−/(i−a₀))²+(j−(j+b₀))²)} (17)

The determining unit 594 determines that the position and direction of the capsule endoscope 3 have been changed when the calculated movement amount M is larger than a specified threshold value and determines that the position and direction have not been changed when the movement amount M is equal to or smaller than the threshold value.

When the determining unit 594 determines that the position and direction of the capsule endoscope 3 have been changed, the estimating unit 595 estimates the position and direction of the capsule endoscope 3 based on the reception strength of the receiving antenna 40.

In the second embodiment, a specified region in an image is set as a template, the movement amount of the template between images is calculated by a block matching method, and the position estimating process is performed only when the movement amount is determined to be large. Thus, it is possible to accelerate the position estimating process and the process of displaying the trajectory calculated based on the results of the position estimating process. Moreover, a region in which the correlation level S has the smallest value is determined by the block matching method as a similar region and the movement amount M is calculated. Thus, it is possible to eliminate system noise and the influence of a delicate movement of the capsule endoscope 3 and to more accurately determine whether the position estimating process will be performed or not.

In the second embodiment, although a movement between images is calculated by the block matching method as an indicator of the change in the position and direction of the capsule endoscope 3, the movement between images can be calculated by an optical flow method.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

The embodiments are merely examples for practicing the present invention, and the present invention is not limited to these embodiments. It is obvious from the above description that various modifications made according to specifications or the like are within the scope of the present invention and various other embodiments can be made within the scope of the present invention.

What is claimed is:

1. A position detecting apparatus of a capsule endoscope, comprising:
a receiving antenna unit that receives a wireless signal transmitted together with an image data signal from the capsule endoscope in a subject via a plurality of receiving antennas;
a first storage unit that stores a first image received by the receiving antennas;
a second storage unit that stores a second image received subsequently to the first image;
a correlation level calculating unit that calculates a correlation level between the first image stored in the first storage unit and the second image stored in the second storage unit;
a determining unit that determines whether a position and/or a direction of the capsule endoscope is changed, based on comparison of the correlation level between the first and second images calculated by the correlation level calculating unit with a specified threshold value;
an estimating unit that estimates the position and/or direction of the capsule endoscope at the time the second image was captured by an imaging unit of the capsule endoscope if the determining unit determines that the correlation level is smaller than the specified threshold value; and
a third storage unit that stores the second image stored in the second storage unit in association with information of the position and/or direction estimated by the estimating unit.

2. The position detecting apparatus of the capsule endoscope according to claim 1, wherein
the correlation level calculating unit calculates a normalized cross-correlation value or a residual sum of squares as the correlation level.

3. The position detecting apparatus of the capsule endoscope according to claim 1, wherein
the correlation level calculating unit calculates a movement amount of a specified region in an image as the correlation level.

4. The position detecting apparatus of the capsule endoscope according to claim 3, wherein
the correlation level calculating unit calculates the movement amount through image processing using a block matching method or an optical flow.

5. The position detecting apparatus of the capsule endoscope according to claim 1, further comprising a fourth storage unit that stores theoretical electric field strengths of the wireless signal received by each of the receiving antennas, according to the position and direction of the capsule endoscope in the subject, wherein
the estimating unit includes:
an electric field strength comparing unit that acquires the theoretical electric field strengths from the fourth storage unit and compares specified values that are calculated from a difference between received electric field strengths of the wireless signal received by each of the receiving antennas and the theoretical electric field strengths; and
a position determining unit that determines the position or the position and direction of the capsule endoscope in which the image data was captured, based on results of the comparison by the electric field strength comparing unit.

6. The position detecting apparatus of the capsule endoscope according to claim 1, wherein
the receiving antenna unit has a sheet shape on which the plurality of receiving antennas are arranged.

7. The position detecting apparatus of the capsule endoscope according to claim 5, further comprising a trajectory calculating unit that calculates a trajectory of the capsule endoscope from the position of the capsule endoscope determined by the position determining unit.

8. A capsule endoscope system comprising:
a capsule endoscope that acquires image data of a subject;
the position detecting apparatus according to claim 1 that receives the image data transmitted from the capsule endoscope and estimates the position and direction of the capsule endoscope when the position and/or direction of the capsule endoscope is determined to be changed; and
an image display unit that acquires the image data and position information of the image data from the position detecting apparatus and displays the acquired image data and position information.

9. The capsule endoscope system according to claim 8, wherein
the image display unit displays the image data and displays a moving trajectory of the capsule endoscope in the subject calculated by the trajectory calculating unit.

10. A non-transitory computer readable recording medium storing an executable position determination program, the position determination program causes a processor of a position detecting apparatus that receives image data transmitted from a capsule endoscope in a subject and estimates a position and a direction of the capsule endoscope in which the received image data was captured, to execute:
acquiring a wireless signal which is transmitted from the capsule endoscope and received by a plurality of receiving antennas of a receiving antenna unit;
extracting an image from the wireless signal received by the receiving antennas;
calculating a correlation level between the extracted image and an image received immediately before the extracted image was received;
determining whether the position and/or direction of the capsule endoscope is changed, based on the calculated correlation level by the correlation level calculating procedure; and
estimating the position and/or direction of the capsule endoscope in which the image was captured if the position and/or direction of the capsule endoscope is determined to be changed by the determining procedure.
